(19) United States
(12) Patent Application Publication
(10) Pub. No.: US 2021/0290276 A1
(43) Pub. Date: Sep. 23, 2021

(54) MEDICAL IMPLANT SYSTEMS AND METHODS OF USE THEREOF

(71) Applicant: Alliance Partners LLC, San Antonio, TX (US)

(72) Inventors: Bruce Riceman, Leander, TX (US); Brad Fredin, Buda, TX (US)

(73) Assignee: Alliance Partners LLC, San Antonio, TX (US)

(21) Appl. No.: 16/825,415

(22) Filed: Mar. 20, 2020

(51) Int. Cl. A61B 17/70; A61B 17/86

(52) U.S. Cl. CPC A61B 17/7059; A61B 17/7044; A61B 2017/00964; A61B 17/866; A61B 17/7091

(57) ABSTRACT

Medical implant systems and methods of use thereof, and, more particularly, bone fixating plating systems and methods of use thereof. The medical implant system includes a plurality of fasteners, a receiving member body having a plurality of openings (capable of receiving one of the plurality of fasteners), and a plurality of locking mechanisms rotatably connected to the receiving member body. Each opening has at least one locking mechanism located nearby. Each locking mechanism can be rotated with an instrument between an open position and a closed position relative to the opening nearby the locking mechanism. Stops prevent over-rotation of the locking mechanism past the open position and the closed position. Each fastener can be rotated with the instrument to secure the fasteners into bone. When the locking mechanism is in the open position, the fastener can be passed through the opening and the instrument can be coupled to the fastener to rotate the fastener. When the locking mechanism is in the closed position, the fastener cannot pass through the opening, the fastener cannot rotate to unsecure the fastener from the bone, and the instrument cannot be coupled to the fastener to rotate the fastener.

### FIELD OF INVENTION

Medical implant systems and methods of use thereof, and, more particularly, bone fixating plating systems and methods of use thereof.

### BACKGROUND OF INVENTION

The spine is the axis of the skeleton on which all of the body parts hang. In humans, the normal spine has seven cervical segments, twelve thoracic segments, five lumbar segments, five sacral segments (which fuse to form the sacrum) and three to five coccygeal segments (which fuse to form the coccyx. The lumbar spine attaches to the pelvis, and in turn is supported by the hip and leg bones. The bony vertebral bodies of the spine are separated by intervertebral discs, which act as joints but allow known degrees of flexion, extension, lateral bending, and axial rotation and translation.

Typical vertebra has a thick anterior bone mass called the vertebral body, with a neural (vertebral) arch that arises from the posterior surface of the vertebral body. The centers of adjacent vertebrae are supported by intervertebral discs. The disc and/or vertebral bodies may be displaced or damaged due to trauma, disease, degenerative defects, or aging over an extended period of time. One result of this displacement or damage to an intervertebral disc or vertebral body may be chronic back pain. In many cases, to alleviate back pain from degenerated or herniated discs, part or all of the disc is removed and may be replaced with an implant that promotes fusion of the remaining bony anatomy.

The success or failure of spinal fusion may depend upon several factors. For instance, the spacer or implant or cage used to fill the space left by the removed disc must be sufficiently strong to support the spine under a wide range of loading conditions. The spacer should also be configured so that it likely to remain in place once it has been positioned in the spine by the surgeon, surgical physician's assistant, etc. Additionally the material used for the spacer should be biocompatible material and should have a configuration that promotes bony ingrowth.

In combination with spacers or cages, a plating system is used to prevent expulsion of the spacer from the spine during the fusion process. These devices, commonly referred to as bone fixation plating systems (i.e., cervical plate), typically include one or more plates and fasteners (typically screws) for aligning and holding vertebrae in a fixed position with respect to one another. Plating systems independent of the spacers have additional complications such as loosening and failure of the hardware. Two common failures are the breakage of the plates, and the backing out of screws into soft tissues of the patient's body.

Cervical cages are used to stabilize the spine during the fusion process. These devices likewise include one or more plates and fasteners (typically screws) for aligning and holding vertebrae in a fixed position with respect to one another. A common failure for corpectomy cages and cervical cages is, like other medical implants, such as plating systems, is the backing out of screws into soft tissues of the patient's body.

Thus, it is important that the medical implant is properly held in place by the fastener.

Moreover, the backing out of the screws is typically a result of the screws failure to achieve a sufficient purchase in the bone, although the stripping of the screws has also been known to cause this problem. Common challenges are that medical implants (such as plating systems and cages) require “carpentry” work to match fit aspects of the vertebral bodies.

To keep the fastener from backing out, a locking (or retention) device is typically utilized. The locking device physically impedes the anterior portion of the screw from rising, which prevents the fastener from rotating (as this would cause the fastener to rise). Once the locking device is in the “locked” position, the fixation mechanism is held in place. Examples of locking mechanisms known in the art are shown in U.S. Pat. No. 8,702,766, issued Apr. 22, 2014 to Mueller; U.S. Pat. No. 8,641,768, issued Feb. 4, 2014, to Duffield et al.; U.S. Pat. No. 9,381,093, issued to Jul. 5, 2016, to Morris et al.; and PCT Patent Appl. Publ. No. WO/2018/237322, filed Aug. 22, 2018, to Swann et al.

There remains a need for improved locking devices on implantable medical devices.

### SUMMARY OF INVENTION

The present invention is a medical implant system (i.e., a bone fixating plating system) that can be held in place by a fastening/fixation mechanism, that is, typically, a screw. To keep the screw from backing out, a locking device is utilized. The locking device physically impedes the anterior portion of the screw from rising, which prevents the screw from rotating (as this would cause the screw to rise).

In general, in one aspect, the invention features a medical implant system. The medical implant system includes a plurality of fasteners. The medical implant system further includes a receiving member body having a plurality of openings. Each of the openings is capable of receiving one of the plurality of fasteners. The medical implant system further includes a plurality of locking mechanisms rotatably connected to the receiving member body. Each opening in the plurality of openings has at least one locking mechanism located nearby. Each of locking mechanism in the plurality of locking mechanisms is capable of being rotated with an instrument between an open position and a closed position relative to the opening nearby the locking mechanism. Each of the fasteners is capable of being rotated with the instrument to secure the fasteners into bone. When the locking mechanism is in the open position, the locking mechanism does not cover any portion of the opening nearby the locking mechanism such that the fastener can be passed through the opening and such that the instrument can be coupled to the fastener to rotate the fastener. When the locking mechanism is in the closed position, a portion of the locking mechanism covers a portion of the opening such that the fastener cannot pass through the opening, such that the fastener cannot rotate to unsecure the fastener from the bone, and such that the instrument cannot be coupled to the fastener to rotate the fastener. The receiving member body has stops that are capable of preventing over-rotation of the locking mechanism past the open position and the closed position.

Implementations of the invention can include one or more of the following features:

The medical implant system can be a bone fixating plating system.

The bone fixating plating system can include a cervical, thoracic, or lumbar plate.

The plurality of fasteners can include a plurality of screws.

The plurality of screws can include screws selected from a group consisting of (a) fixed-angle screws, (b) variable-angle screws, (c) self-drilling screws, (d) self-tapping screws, and (e) combinations thereof.

The receiving member body can have a thickness of 0.5 mm to 4 mm and a width of 5 mm to 40 mm.

The receiving member body can be selected from a group consisting of a one level plate, a two level plate, a three level plate, a four level plate, and a five level plate.

The fastener can include a material selected from a group consisting of metal alloys, polymers, ceramics, and composites thereof.

The receiving member body can have a lordotic and medial/lateral curvature.

The receiving member body can have radial cut sections on face of the receiving member body such that the receiving member body is capable to be bent utilizing a bending instrument for positioning the plurality of openings.

The distance from the end of the receiving member body to the center of at least one of the openings in the plurality of openings can be at most 4 mm.

The receiving member body can have one or more windows to provide visualization through the receiving member body.

The distance from the center of each of the openings to a closest wall of the receiving member body forming one of the one or more windows can be at most 2 mm.

The receiving member body can have at least one pair of tapered surfaces that are capable of mating with a location/guide instrument.

Each of the fasteners in the plurality of fasteners and each of the locking mechanisms in the plurality of locking mechanisms can have the same shape-coupling portion that provides for the instrument to couple with each of the fasteners and the locking mechanisms.

In general, in another aspect, the invention features a method that includes a method that includes the step of selecting an above-described medical implant system having a plurality of fasteners, a receiving member body having a plurality of openings, and a plurality of locking mechanisms. The method further includes the step of, while a first locking mechanism is in an open position relative to a nearby first opening in the plurality of openings, inserting a first fastener in the plurality of fasteners through the nearby first opening such that the first fastener is received into the receiving member body. The method further includes the step of coupling an instrument to the first fastener. The method further includes the step of utilizing the instrument to rotate the first fastener to secure the first fastener and the receiving body to bone. The method further includes the step of uncoupling the instrument from the first fastener. The method further includes the step of coupling the instrument to the first locking mechanism. The method further includes the step of utilizing the instrument to rotate the first locking mechanism from the open position to a closed position, wherein a portion of the first locking mechanism covers a portion of the first opening such that the first fastener cannot pass through the first opening, and such that the first fastener cannot rotate to unsecure the first fastener from the bone. The method further includes the step of decoupling the instrument from the first locking mechanism. The method further includes the step of repeating the above-described steps for additional fasteners in the plurality of fasteners to secure the receiving member body.

Implementations of the invention can include one or more of the following features:

The method can further include utilizing a fixation pin to secure the receiving member body and hold the receiving member body in place. The method can further include, after securing the receiving member body utilizing the plurality of fasteners, removing the fixation pin.

The method can further include coupling the instrument to the first locking mechanism, utilizing the instrument to rotate the first locking mechanism from the closed position to the open position closed position;, and decoupling the instrument from the first locking mechanism. The method can further include coupling the instrument to the first fastener, utilizing the instrument to rotate the first fastener to remove the first fastener from the bone, uncoupling the instrument from the first fastener, and removing the first fastener from the first locking mechanism. The method can further include repeating the above-described steps for additional fasteners in the plurality of fasteners to unsecure the receiving member body.

The medical implant system can be a bone fixating plating system.

The bone fixating plating system can include a cervical, thoracic, or lumbar plate.

The plurality of fasteners can include a plurality of screws.

The plurality of screws can include screws selected from a group consisting of (a) fixed-angle screws, (b) variable-angle screws, (c) self-drilling screws, (d) self-tapping screws, and (e) combinations thereof.

The receiving member body can have a thickness of 0.5 mm to 4 mm and a width of 5 mm to 40 mm.

The receiving member body can be selected from a group consisting of a one level plate, a two level plate, a three level plate, a four level plate, and a five level plate.

The fastener can include a material selected from a group consisting of metal alloys, polymers, ceramics, and composites thereof.

The receiving member body can have a lordotic and medial/lateral curvature.

The receiving member body can have radial cut sections on face of the receiving member body. The method can further include utilizing a bending instrument to bend the receiving member body to position the plurality of openings.

The distance from the end of the receiving member body to the center of at least one of the openings in the plurality of openings can be at most 4 mm.

The receiving member body can have one or more windows to provide visualization through the receiving member body.

The distance from the center of each of the openings to a closest wall of the receiving member body forming one of the one or more windows can be at most 2 mm.

The receiving member body can have at least one pair of tapered surfaces. The method can further include mating with a location/guide instrument utilizing the at least one pair of the tapered surfaces.

Each of the fasteners in the plurality of fasteners and each of the locking mechanisms in the plurality of locking mechanisms can have the same shape-coupling portion that provides for the instrument to couple with each of the fasteners and the locking mechanisms.

The foregoing has outlined rather broadly the features and technical advantages of the invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims.

It is also to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 is an isometric view of a medical implant of the present invention with the locking mechanisms in the locked position.

FIGS. 2A-2D are, respectively, anterior, superior/inferior, sagittal, and posterior view of the medical implant shown in FIG. 1. (For the features of the medical implant shown, which is an anterior cervical medical implant, the anterior view is the front view and the sagittal view is a side view, etc., in the normal orientation of use. Hence, unless otherwise indicated, terms like “anterior,” “posterior,” “sagittal,” “superior,” and “inferior” are describing front, back, side, top, and bottom, respectively for the normal orientation of use of an anterior cervical medical implant.

FIG. 3A is an anterior view of the anterior medical implant shown in FIG. 1 in which one of the locking mechanisms is in the unlocked position.

FIG. 3B is a magnified portion of the medical implant shown in FIG. 3A.

FIG. 4A is an anterior view of the medical implant shown in FIG. 1 in which some of the locking mechanisms are in the unlocked position.

FIG. 4B is a cross-sectional view of medical implant taken from cross-section B-B′ shown in FIG. 4A.

FIG. 4C is a cross-sectional view of medical implant taken from cross-section A-A′ shown in FIG. 4A.

FIG. 5A is an anterior view of the medical implant shown in FIG. 1 in which some of the locking mechanisms are in the unlocked position and others are in the locked position.

FIG. 5B is a cross-sectional view of medical implant taken from cross-section C-C′ shown in FIG. 5A.

### DETAILED DESCRIPTION

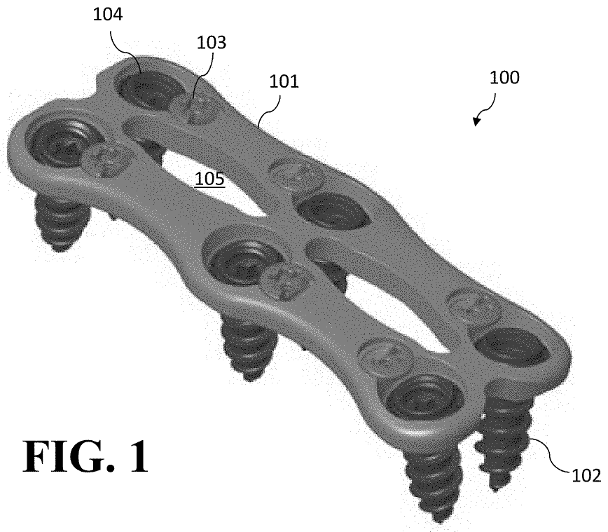

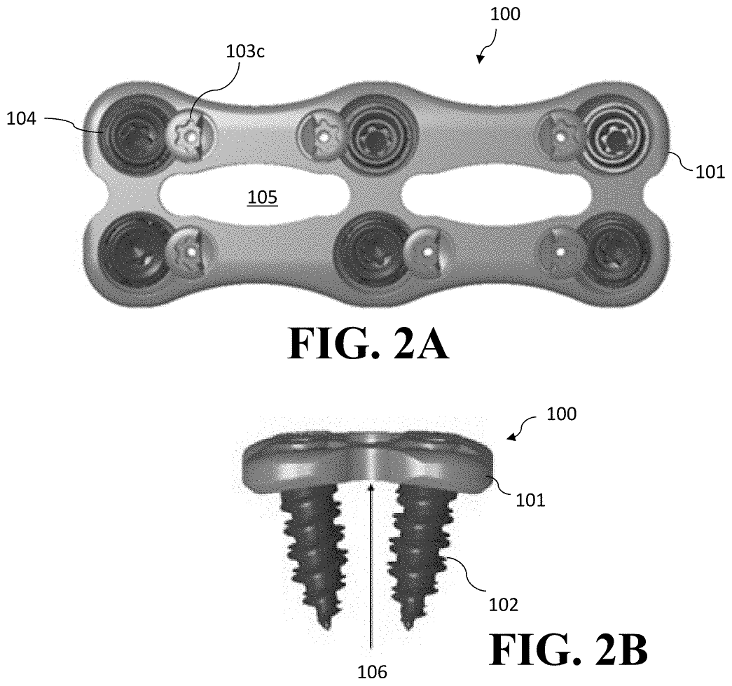

The technology relates to a medical implant system and method of use thereof, and, more particularly, a bone fixating plating system and method of use thereof. Referring to the figures, FIG. 1 is an isometric view of an anterior plate assembly 100 with a plate portion 101 and a plurality of locking mechanisms 103c. As shown in FIG. 1, each of locking mechanism is in the locked (or closed) position. (For clarification, when a locking mechanism is in the locked/closed position, it will be identified as locking mechanism 103c, and when a locking mechanism is in the unlocked/open position, it will be identified as locking mechanism 103u). When the locking mechanism 103c is in the “locked” (or “closed”) position, the fastening/fixation mechanism 102 is held in place. The locking mechanism 103c keeps each of the bone fasteners 102 (such as screws) from backing out of their respective openings 104. The locking mechanism 103c physically impedes the anterior portion of the fastener 102 (i.e., screw head) from rising, which prevents the fastener 102 from rotating (as this would cause the fastener 102 to rise). (The fastener/fixation mechanism can be inserted and removed when the locking mechanism 103u is in the “unlocked” (or “open”) position). FIGS. 2A-2D show, respectively, anterior, superior/inferior, sagittal, and posterior views of anterior plate assembly 100. The locking mechanisms are located in plate portion 101 to allow for a higher fastener insertion angle in a cephalad/caudal direction and to minimize the thickness of the plate portion 101.

Plate portion 101 has lordotic and medial/lateral curvature 106 built-in to better match the patient's anatomy.

Plate portion 101 has radial cut sections 107 on the posterior side that mate with a plate bender instrument for proper bending to occur between the openings 104 (and not over openings 104).

Plate portion 101 further has windows 105.

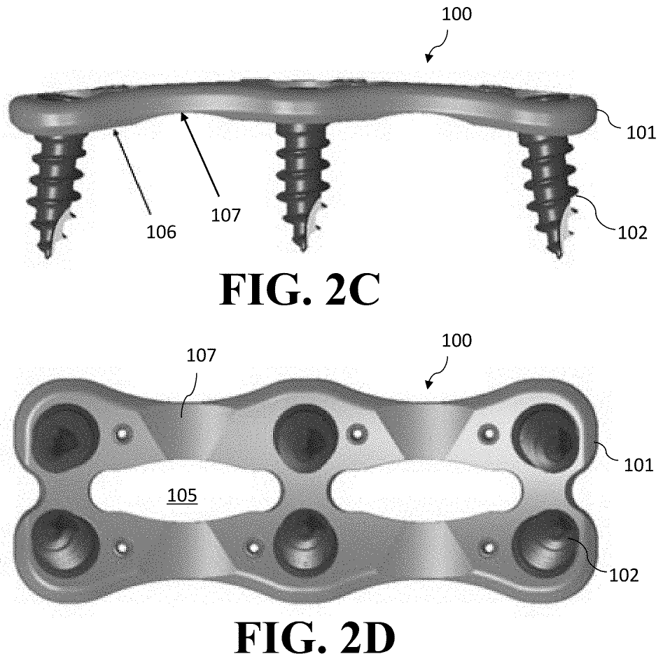

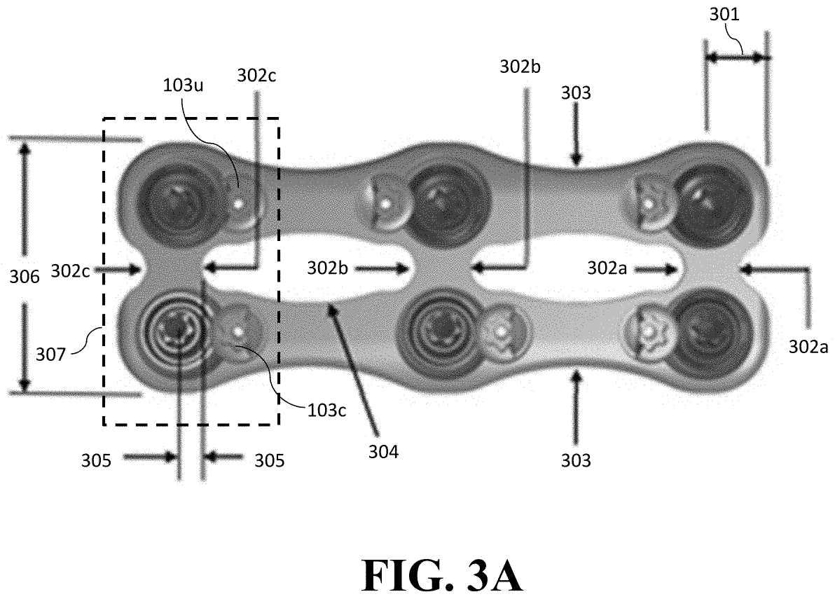

FIG. 3A is an anterior view of anterior plate assembly 100 in which one of the locking mechanisms 103 in FIG. 1 has been moved to an unlocked position, which unlocked mechanism is shown as locking mechanism 103o in FIG. 3A. As shown in FIG. 3A, plate portion 101 has a distance 301 that is generally less than 4.0 mm between the end of the plate portion 101 to the center of the fastener 102 to maximize the distance from the adjacent vertebral body.

Plate portion 101 has features (shown by arrow pairs 302a, 302b, and 303c) at each cervical level that allows for a temporary plate fixation pin to be inserted on the mid-line of plate portion 101.

Plate portion 101 has narrow sections 303 between the fasteners 102 to increase visualization of the disk space at each cervical level. Plate portion 101 also has wide openings 304 to allow for visualization through windows 105 of the graft at each cervical level.

Plate portion 101 has a distance that is generally less than 2.0 mm between the center of the head of fasteners 102 to the end of the graft windows 105 to maximize visualization to aid with placement of fasteners 102.

Plate portion 101 has a narrow overall width for a better fit on the vertebral bodies and allow for smaller cervical incisions.

Box 307 surrounds a portion of anterior plate assembly 100 that includes locking mechanism 103u (which is locking mechanism in the unlocked position) and another locking mechanism 103c (which is a locking mechanism in the locked position). The portion of anterior plate assembly 100 in box 307 is shown in FIG. 3B (magnified).

As shown in FIG. 3B, the locking mechanisms (locking mechanism 103u in the open position and locking mechanism 103c in the close position) have a portion 309 that is the same as portion 308 on fasteners 102, so the same device (such as a screwdriver or other rotational device) can be used to rotated the locking mechanisms and the fasteners 102. Having the same device to rotate the fasteners 102 and the locking mechanisms reduces surgical time by eliminating an extra instrument exchange for the surgeon/practitioner.

Plate portion 101 further has positive stops 310 that limit the amount the locking mechanism can be rotating for ease of use by the surgeon/practitioner.

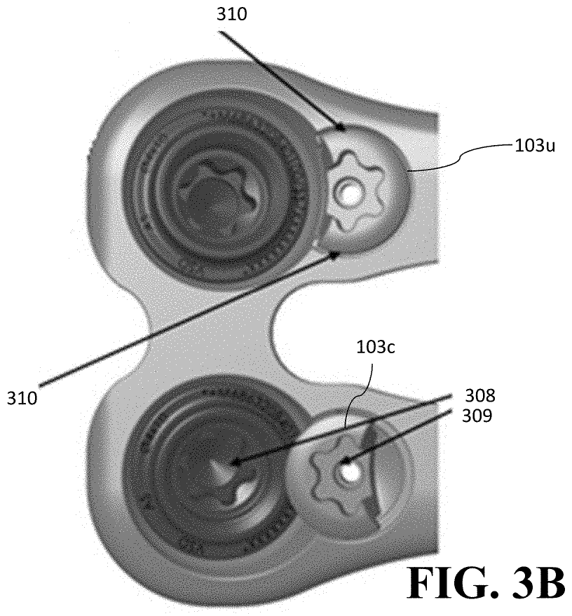

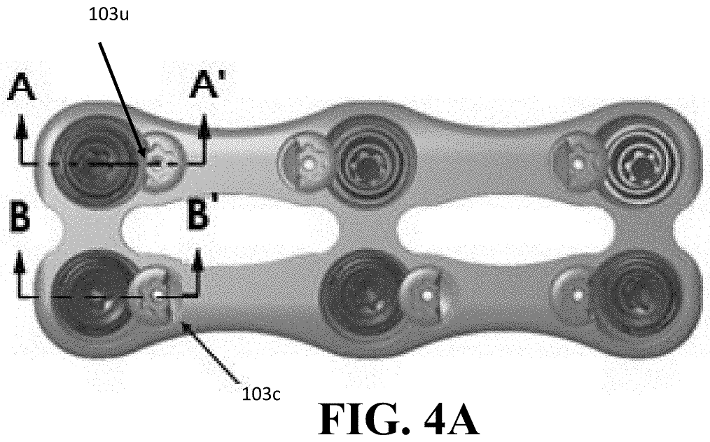

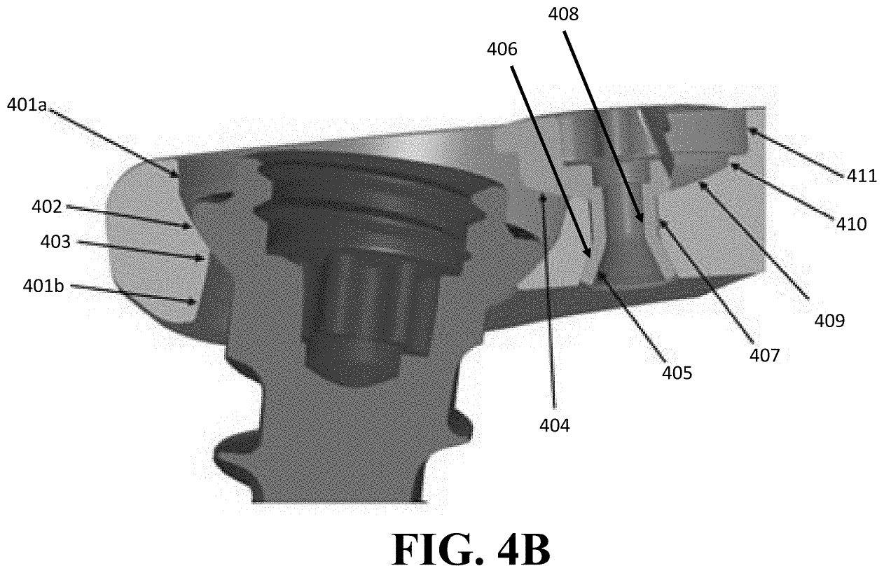

FIG. 4A is an anterior view of the anterior plate assembly 100 in which some of the locking mechanisms are in the unlocked position. FIG. 4B-4C are respective cross-sectional views of anterior plate assembly 100 from cross-sections B-B′ and A-A′ shown in FIG. 4A. FIGS. 4B-4C further show the locking mechanisms interactions with plate portion 101.

FIG. 4B shows a cross-section B-B′ across locking mechanism 103c (a locking mechanism in the locked position). Plate portion 101 has surfaces 401a-401b that limit the angles in which fasteners 102 can be inserted into openings 104. Plate portion has a spherical radius 402 that matches the spherical radius of the fasteners 102 and of the instruments (i.e., rotational device), which allow the fasteners 102 and instruments to be inserted at various angles.

Plate portion 101 has a cylindrical bore 403 that prevents the fasteners 102 from going opening 104 and through plate portion 101, limits the angle that the fasteners 102 and instruments can be inserted, and limits movement of the fasteners 102 after being implanted.

The locking mechanism has a portion 404 that partially covers opening 104 when turned to the locked position. Portion 404 prevents the fastener 102 from backing-out of plate portion 101.

Locking mechanism is pressed during assembly so material 405 is moved outward diametrically to a mating feature 406 within plate creating rotational friction and so the locking mechanism is not able to dissociate from the plate portion 101 but can rotate when turned.

The locking mechanism and plate portion 101 have, respectively, mating diametrical features 407-408 that control the location to each other.

Locking mechanism and plate portion 101 have spherical features 409 that aid with rotation and controls the distance between the fastener head and locking mechanism. Plate portion 101 has an internal diameter 410 that mates with the locking mechanism external diameter and a clearance fit portion 411 that permits that locking mechanism to be rotated into the unlocked/open position so that portion 404 of the locking mechanism no longer covers opening 104. FIG. 4C shows a cross-section A-A′ across locking mechanism 103c (a locking mechanism in the unlocked position). Again, it is only when the locking mechanism is turned to the locked position the result is the locking mechanism preventing fastener 102 from backing-out of opening 104 in plate portion 101. When the locking mechanism in turned to the unlocked position (as shown in FIG. 4C), opening 104 now is opened and there is a clearance 412 that allows for the insertion and removal of fastener 102 and the instruments.

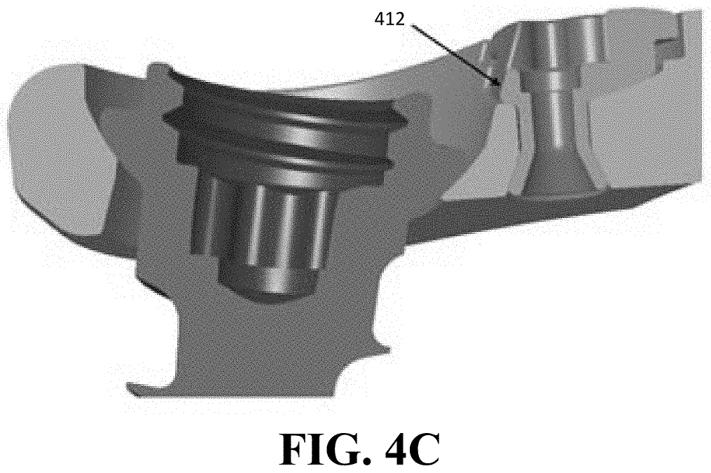

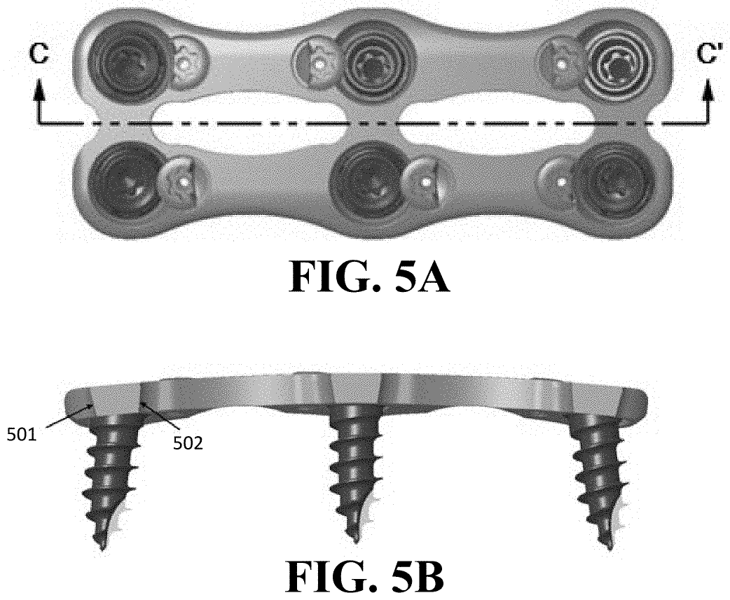

FIG. 5A is an anterior view of anterior plate assembly 100 in which some of the locking mechanisms are in the unlocked position and others are in the locked position. FIG. 5B is a cross-sectional view anterior plate assembly 100 from cross-section C-C′ shown in FIG. 5A. FIG. 5B shows that plate portion 101 has two tapered surfaces 501-502 at each level, which mate with instruments for positive attachment and/or locational purposes for guides.

### Plate Variations

While the figures refer to medical implant that is an anterior cervical plate, the medical implant can be otherwise used, such as in other plates.

This system can be utilized for anterior screw fastening/fixation to the cervical spine and can include a variety of shapes and sizes of plates (bone plates) and fasteners (screws).

### Components and Fasteners

The components can be manufactured from any implant grade metal alloy, such as titanium alloy (Ti 6A1 4V ELI as described by ASTM F136), as well as ceramic, polymer, or composites thereof. In some embodiments of the present invention, the sterile components are provided. In other embodiments of the present invention, the non-sterile components are provided, which will then need to be steam sterilized by a hospital or surgeon prior to use.

Additional features and benefits of the present invention include:

Typical thickness and width of the plate is 0.5-4.0 mm×15-40 mm.

The length of the plate can include one, two, three, four, and five level plates. (Plate, such as cervical plates, are generally referred to by the number of levels that they overlie, wherein the word “level” refers to the number of intervening intervertebral spaces that are spanned. Thus, for example, a three level cervical plate would span the four vertebrae beyond and between the three intervertebral spaces).

Serrated, textures, and/or knurled under surfaces for grip during fastener placement/delivery.

Fasteners can be in fixed or variable angle and/or self-drilling/self-tapping, which allow the surgeon/practitioner to create a variety of constructs. While the size can be varied, ∅4.0 to ∅4.5 mm fasteners (shank diameter) can be utilized in anterior cervical plate assemblies. A larger fastener ∅5.5 mm fastener (shank diameter) can be utilized in linear cervical plate that have larger openings compatible for such larger fasteners. Diameters of the fasteners (shank diameters) can be any size, such as between ∅3.5-6.5 mm.

### Uses

Embodiments of the invention can be used for anterior intervertebral screw fastening/fixation of the cervical spine at levels C2-T1. Embodiments can be used in the temporary stabilization of the anterior spine during the development of cervical spinal fusion in patients with the following indications, including, but not limited to:

Degenerative Disc Disease (as defined by neck pain of discogenic origin with degeneration of the disc confirmed by patient history and radiographic studies)

Trauma (including fractures)

Tumors

Deformities or curvatures (including kyphosis, lordosis, or scoliosis)

Pseudoarthrosis

Failed previous fusions

Spondylolisthesis

Spinal stenosis

Revision surgery

Embodiments of the invention can be utilized during procedures known in the art utilizing plating systems. For example, a surgeon/practitioner can perform preoperative planning, which includes the selection of a medical implant, such as the anterior plate assembly 100 shown in FIG. 1 Because the plate has a narrow overall width, it can for a better fit on the vertebral bodies and allow for smaller cervical incisions. The plate also can be selected to increase visibility of the disc space at each cervical level and to allow visualization of the graft for each cervical level.

The surgeon/practitioner can perform the procedures and in the steps that include the plating system the surgeon/practitioner can first adjust the plating system, such as by utilizing a plate bender instrument for proper bending to occur between the fastener openings and not the fastener openings.

The surgeon/practitioner can then position the plating system. Optionally, a temporary fixation pin can be inserted on the mid-line of the plate to hold it in position while the plate is being secured utilizing the fasteners.

The surgeon/practitioner can then utilize an instrument (a rotating device) that has a connector that can couple to the fastener head and can also couple to the locking mechanism. The locking mechanism is set in the open/unlocked position such that the surgeon/practitioner can insert the fastener through the opening and secure the fastener into bone. Once the fastener is secured, the surgeon/practitioner can then rotate the locking mechanism utilizing the same instrument and set the locking mechanism in the locked/closed position. The positive stops on the plate prevent the over-rotation of the locking mechanism. The surgeon repeats these steps into the plate is fully secured with a plurality of fasteners through a corresponding plurality of openings of the plate. Once secured, the surgeon practitioner can remove the mid-line temporary fixation pin (if one had optionally be utilized).

In a separate procedure, the surgeon/practitioner can remove the plate by unfastening it. Again, using the same tool, the surgeon/practitioner can rotate the locking mechanism into the unlocked/open position and then remove the fastener.

The disclosures of all patents, patent applications, and publications cited herein are hereby incorporated herein by reference in their entirety, to the extent that they provide exemplary, procedural, or other details supplementary to those set forth herein. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention.

While embodiments of the invention have been shown and described, modifications thereof can be made by one skilled in the art without departing from the spirit and teachings of the invention. The embodiments described and the examples provided herein are exemplary only and are not intended to be limiting. Many variations and modifications of the invention disclosed herein are possible and are within the scope of the invention. Accordingly, the scope of protection is not limited by the description set out above.

What is claimed is:

1. A medical implant system comprising:
(a) a plurality of fasteners;
(b) a receiving member body having a plurality of openings, wherein each of the openings is capable of receiving one of the plurality of fasteners;
(c) a plurality of locking mechanisms rotatably connected to the receiving member body, wherein
(i) each opening in the plurality of openings has at least one locking mechanism located nearby,
(ii) each of locking mechanism in the plurality of locking mechanisms is capable of being rotated with an instrument between an open position and a closed position relative to the opening nearby the locking mechanism;
(iii) each of the fasteners is capable of being rotated with the instrument to secure the fasteners into bone;
(iv) when the locking mechanism is in the open position, the locking mechanism does not cover any portion of the opening nearby the locking mechanism such that the fastener can be passed through the opening and such that the instrument can be coupled to the fastener to rotate the fastener;
(v) when the locking mechanism is in the closed position, a portion of the locking mechanism covers a portion of the opening such that the fastener cannot pass through the opening, such that the fastener cannot rotate to unsecure the fastener from the bone, and such that the instrument cannot be coupled to the fastener to rotate the fastener; and
(vi) the receiving member body has stops that are capable of preventing over-rotation of the locking mechanism past the open position and the closed position.

2. The medical implant system of claim 1, wherein the medical implant system is a bone fixating plating system.

3. The medical implant system of claim 2, wherein the bone fixating plating system comprises a cervical, thoracic, or lumbar plate.

4. The medical implant system of claim 1, wherein the plurality of fasteners comprise a plurality of screws.

5. The medical implant system of claim 4, wherein the plurality of screws comprise screws selected from a group consisting of (a) fixed-angle screws, (b) variable-angle screws, (c) self-drilling screws, (d) self-tapping screws, and (e) combinations thereof.

6. The medical implant system of claim 1, wherein the receiving member body has a thickness of 0.5 mm to 4 mm and a width of 5 mm to 40 mm.

7. The medical implant system of claim 1, wherein the receiving member body is selected from a group consisting of a one level plate, a two level plate, a three level plate, a four level plate, and a five level plate.

8. The medical implant system of claim 1, wherein the fastener comprises a material selected from a group consisting of metal alloys, polymers, ceramics, and composites thereof.

9. The medical implant system of claim 1, wherein the receiving member body has a lordotic and medial/lateral curvature.

10. The medical implant system of claim 1, wherein the receiving member body has radial cut sections on face of the receiving member body such that the receiving member body is capable to be bent utilizing a bending instrument for positioning the plurality of openings.

11. The medical implant system of claim 1, wherein distance from the end of the receiving member body to the center of at least one of the openings in the plurality of openings is at most 4 mm.

12. The medical implant system of claim 1, wherein the receiving member body has one or more windows to provide visualization through the receiving member body.

13. The medical implant system of claim 12, wherein distance from the center of each of the openings to a closest wall of the receiving member body forming one of the one or more windows is at most 2 mm.

14. The medical implant system of claim 1, wherein the receiving member body has at least one pair of tapered surfaces that are capable of mating with a location/guide instrument.

15. The medical implant system of claim 1, wherein each of the fasteners in the plurality of fasteners and each of the locking mechanisms in the plurality of locking mechanisms has the same shape-coupling portion that provides for the instrument to couple with each of the fasteners and the locking mechanisms.

16. A method comprising the steps of:
(a) selecting a medical implant system of claim 1 having a plurality of fasteners, a receiving member body having a plurality of openings, and a plurality of locking mechanisms;
(b) while a first locking mechanism is in an open position relative to a nearby first opening in the plurality of openings, inserting a first fastener in the plurality of fasteners through the nearby first opening such that the first fastener is received into the receiving member body;
(c) coupling an instrument to the first fastener;
(d) utilizing the instrument to rotate the first fastener to secure the first fastener and the receiving body to bone;
(e) uncoupling the instrument from the first fastener;
(f) coupling the instrument to the first locking mechanism;
(g) utilizing the instrument to rotate the first locking mechanism from the open position to a closed position, wherein a portion of the first locking mechanism covers a portion of the first opening such that the first fastener cannot pass through the first opening, and such that the first fastener cannot rotate to unsecure the first fastener from the bone;
(h) decoupling the instrument from the first locking mechanism; and
(i) repeating steps (b)-(h) for additional fasteners in the plurality of fasteners to secure the receiving member body.

17. The method of claim 16, wherein the method further comprises:
(a) before step (b), utilizing a fixation pin to secure the receiving member body and hold the receiving member body in place during steps (b)-(h); and
(b) after the completion of step (i), removing the fixation pin.

18. The method of claim 16, wherein the method further comprises:
(a) coupling the instrument to the first locking mechanism;
(b) utilizing the instrument to rotate the first locking mechanism from the closed position to the open position closed position;
(c) decoupling the instrument from the first locking mechanism;
(d) coupling the instrument to the first fastener;
(e) utilizing the instrument to rotate the first fastener to remove the first fastener from the bone;
(e) uncoupling the instrument from the first fastener;
(f) removing the first fastener from the first locking mechanism; and
(g) repeating steps steps (a)-(g) for additional fasteners in the plurality of fasteners to unsecure the receiving member body.

19. The method of claim 16, wherein the medical implant system is a bone fixating plating system.

20. The method of claim 19, wherein the bone fixating plating system comprises a cervical, thoracic, or lumbar plate.

21. The method of claim 16, wherein the plurality of fasteners comprise a plurality of screws.

22. The method of claim 21, wherein the plurality of screws comprise screws selected from a group consisting of (a) fixed-angle screws, (b) variable-angle screws, (c) self-drilling screws, (d) self-tapping screws, and (e) combinations thereof.

23. The method of claim 16, wherein the receiving member body has a thickness of 0.5 mm to 4 mm and a width of 5 mm to 40 mm.

24. The method of claim 16, wherein the receiving member body is selected from a group consisting of a one level plate, a two level plate, a three level plate, a four level plate, and a five level plate.

25. The method of claim 16, wherein the fastener comprises a material selected from a group consisting of metal alloys, polymers, ceramics, and composites thereof.

26. The method of claim 16, wherein the receiving member body has a lordotic and medial/lateral curvature.

27. The method of claim 16, wherein
(a) the receiving member body has radial cut sections on face of the receiving member body; and
(b) the method further comprises utilizing a bending instrument to bend the receiving member body to position the plurality of openings.

28. The method of claim 16, wherein distance from the end of the receiving member body to the center of at least one of the openings in the plurality of openings is at most 4 mm.

29. The method of claim 16, wherein the receiving member body has one or more windows to provide vsualization through the receiving member body.

30. The method of claim 29, wherein distance from the center of each of the openings to a closest wall of the receiving member body forming one of the one or more windows is at most 2 mm.

31. The method of claim 16, wherein
(a) the receiving member body has at least one pair of tapered surfaces; and
(b) the method further comprises mating with a location/guide instrument utilizing the at least one pair of the tapered surfaces.

32. The method of claim 16, wherein each of the fasteners in the plurality of fasteners and each of the locking mechanisms in the plurality of locking mechanisms has the same shape-coupling portion that provides for the instrument to couple with each of the fasteners and the locking mechanisms.
